(19) United States
(12) Patent Application Publication
(10) Pub. No.: US 2010/0036014 A1
(43) Pub. Date: Feb. 11, 2010

(54) STABILIZED ANTIMICROBIAL POLYCARBONATE COMPOSITIONS

(75) Inventors: John J. Charles, Upper St. Clair, PA (US); Michael A. Gerace, Slovan, PA (US)

Correspondence Address: BAYER MATERIAL SCIENCE LLC, 100 BAYER ROAD, PITTSBURGH, PA, 15205, US

(73) Assignee: BAYER MATERIALSCIENCE LLC

(21) Appl. No.: 12/186,777

(22) Filed: Aug. 6, 2008

(51) Int. Cl. A61F 2/00

(52) U.S. Cl. 523/105

(57) ABSTRACT

The present invention provides a stabilized thermoplastic molding composition comprising an aromatic polycarbonate resin and from about 0.1 to about 40 percent, based on the weight of the composition, of a silver sodium zirconium phosphate antimicrobial compound of the formula (I)

NaₓH_{y}Ag_{z}Zr₂(PO₄)₃ (I)

wherein the composition has a melt volume rate according to ASTM 1238 within about 40% of the melt volume rate of a comparable composition not containing the antimicrobial compound. The composition of the present invention may find use in preparing useful articles, such as medical articles, by any method of thermoplastic processing including injection molding and extrusion.

### FIELD OF THE INVENTION

The present invention relates, in general, to a thermoplastic, molding composition and, more specifically, to a stabilized antimicrobial polycarbonate composition having good color retention and melt rheology.

### BACKGROUND OF THE INVENTION

Antibiotic resin compositions are known to those skilled in the art. Such compositions typically contain inorganic, silver containing antimicrobial compounds, e.g., U.S. Pat. Nos. 4,938,955; 5,698,212; 5,698,229; and 5,827,524.

JP9216999 teaches an antimicrobial preparation made from a glass composition containing 40 to 55 mol % P₂O₅, 35 to 45 mol % ZnO, 5 to 15 mol % Al₂O₃, 1 to 10 mol % B₂O₃, and 0.3 to 1.0 wt. % Ag₂O, based on 100 parts by weight of the glass composition. This preparation is said to stabilize the antimicrobial activity and cause no significant discoloration or deterioration of the impact resistance of a polycarbonate resin while also improving durability.

JP6240125 discloses a composition that is said to be excellent in thermal stability and impact resistance which is made by blending a thermoplastic resin consisting essentially of a polycarbonate resin with antimicrobial calcium phosphates containing antimicrobial metallic ions supported thereon and a phosphorus-based stabilizer.

U.S. Pat. No. 6,187,456, issued to Lever, teaches an improved method of inhibiting undesirable discoloring of plastic articles containing silver-based antimicrobials. The method of Lever requires very low amounts of acid scavengers or stabilizers such as aluminum-magnesium hydroxycarbonate, otherwise known as hydrotalcite (and not a zinc-based compound). Such hydrotalcites are said to substantially prohibit the generation of unwanted aesthetically displeasing colors.

One of the problems inherent in the art is that inorganic antimicrobial additives included to protect polycarbonate from microbial or fungal attack when added at commercially viable concentrations or higher, result in materials with reduced melt rheology and poor color retention during melt processing.

Therefore, a need exists in the art for thermoplastic resins containing inorganic antimicrobial additives to protect polycarbonate from microbial or fungal attack which will provide materials with increased melt rheology and good color retention during melt processing when the antimicrobials are added at commercially viable concentrations or higher.

### SUMMARY OF THE INVENTION

Accordingly, the present invention provides compositions based on silver sodium zirconium phosphate of the following composition:

NaₓH_{y}Ag_{z}Zr₂(PO₄)₃

where x+y+z=1.

Surprisingly, the inventive polycarbonate compositions exhibit excellent melt stability and color retention while eliminating foaming and retaining antimicrobial behavior when processed at elevated temperatures, even with significantly increased levels of the silver antimicrobial additive included. These and other advantages and benefits of the present invention will be apparent from the Detailed Description of the Invention herein below.

### DETAILED DESCRIPTION OF THE INVENTION

The present invention will now be described for purposes of illustration and not limitation. Except in the operating examples, or where otherwise indicated, all numbers expressing quantities, percentages and so forth in the specification are to be understood as being modified in all instances by the term “about.” Equivalent weights and molecular weights given herein in Daltons (Da) are number average equivalent weights and number average molecular weights respectively, unless indicated otherwise.

The present invention provides a stabilized thermoplastic molding composition comprising an aromatic polycarbonate resin and from 0.1 to 40 weight percent, based on the weight of the composition, of a silver sodium zirconium phosphate antimicrobial compound of the formula (I)

aₓH_{y}Ag_{z}Zr₂(PO₄)₃ (I)

wherein x+y+z=1, and wherein the composition has a melt volume rate according to ASTM 1238 within 40% of the melt volume rate of a comparable composition not containing the antimicrobial compound.

The present invention further provides a process for the production of a stabilized molding composition comprising combining an aromatic polycarbonate resin and from 0.1 to 40 weight percent, based on the weight of the composition, of a silver sodium zirconium phosphate antimicrobial compound of the formula (I)

aₓH_{y}Ag_{z}Zr₂(PO₄)₃ (I)

wherein x+y+z=1, and
wherein the composition has a melt volume rate according to ASTM 1238 within about 40% of the melt volume rate of a comparable composition not containing the antimicrobial compound.

Suitable polycarbonate resins for preparing the composition of the present invention are homopolycarbonates and copolycarbonates, both linear or branched resins and mixtures thereof.

The polycarbonates have a weight average molecular weight of preferably 10,000 to 200,000, more preferably 20,000 to 80,000 and their melt flow rate, per ASTM D-1238 at 300° C., is preferably 1 to 65 g/10 min., more preferably 2 to 35 g/10 min. They may be prepared, for example, by the known diphasic interface process from a carbonic acid derivative such as phosgene and dihydroxy compounds by polycondensation (See, German Offenlegungsschriften 2,063,050; 2,063,052; 1,570,703; 2,211,956; 2,211,957 and 2,248,817; French Patent 1,561,518; and the monograph by H. Schnell, “Chemistry and Physics of Polycarbonates”, Interscience Publishers, New York, N.Y., 1964).

In the present context, dihydroxy compounds suitable for the preparation of the polycarbonates of the invention conform to the structural formulae (1) or (2) below.

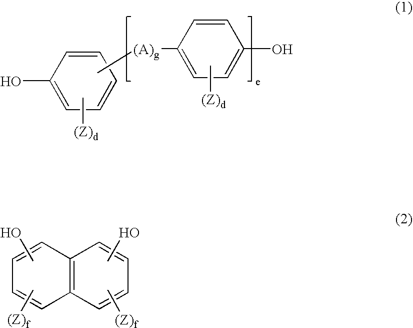

wherein
A denotes an alkylene group with 1 to 8 carbon atoms, an alkylidene group with 2 to 8 carbon atoms, a cycloalkylene group with 5 to 15 carbon atoms, a cycloalkylidene group with 5 to 15 carbon atoms, a carbonyl group, an oxygen atom, a sulfur atom, —SO— or —SO₂ or a radical conforming to

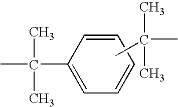

e and g both denote the number 0 to 1:
Z denotes F, Cl, Br or C₁-C₄-alkyl and if several Z radicals are substituents in one aryl radical, they may be identical or different from one another;
d denotes an integer of from 0 to 4; and
f denotes an integer of from 0 to 3.

Among the dihydroxy compounds useful in the practice of the invention are hydroquinone, resorcinol, bis-(hydroxyphenyl)-alkanes, bis-(hydroxy-phenyl)-ethers, bis-(hydroxyphenyl)-ketones, bis-(hydroxy-phenyl)-sulfoxides, his-(hydroxyphenyl)-sulfides, bis-(hydroxyphenyl)-sulfones, and α,α-bis-(hydroxyphenyl)-diisopropylbenzenes, as well as their nuclear-alkylated compounds. These and further suitable aromatic dihydroxy compounds are described, for example, in U.S. Pat. Nos. 5,401,826, 5,105,004; 5,126,428; 5,109,076; 5,104,723; 5,086,157; 3,028,356; 2,999,835; 3,148,172; 2,991,273; 3,271,367; and 2,999,846, the contents of which are incorporated herein by reference.

Further examples of suitable bisphenols are 2,2-bis-(4-hydroxyphenyl)-propane (bisphenol A), 2,4-bis-(4-hydroxyphenyl)-2-methyl-butane, 1,1 -bis-(4-hydroxyphenyl)-cyclohexane, α,α′-bis-(4-hydroxy-phenyl)-p-diisopropylbenzene, 2,2-bis-(3-methyl-4-hydroxyphenyl)-propane, 2,2-bis-(3-chloro-4-hydroxyphenyl)-propane, 4,4′-dihydroxy- diphenyl, bis-(3,5-dimethyl-4-hydroxyphenyl)-methane, 2,2-bis-(3,5-dimethyl-4-hydroxyphenyl)-propane, bis-(3,5-dimethyl-4-hydroxyphenyl)-sulfide, bis-(3,5-dimethyl-4-hydroxy-phenyl)-sulfoxide, bis-(3,5-dimethyl-4-hydroxyphenyl)-sulfone, dihydroxy-benzophenone, 2,4-bis-(3,5-dimethyl-4-hydroxyphenyl)-cyclohexane, α,α′-bis-(3,5-dimethyl-4-hydroxyphenyl)-p-diisopropyl-benzene and 4,4′-sulfonyl diphenol.

Examples of particularly preferred aromatic bisphenols are 2,2-bis-(4-hydroxyphenyl)-propane, 2,2-bis-(3,5-dimethyl-4-hydroxyphenyl)-propane, 1,1-bis-(4-hydroxyphenyl)-cyclohexane and 1,1-bis-(4-hydroxy-phenyl)-3,3,5-trimethylcyclohexane. The most preferred bisphenol is 2,2-bis-(4-hydroxyphenyl)-propane (bisphenol A).

The polycarbonates of the invention may entail in their structure units derived from one or more of the suitable bisphenols.

Among the resins suitable in the practice of the invention are phenolphthalein-based polycarbonate, copolycarbonates and terpoly-carbonates such as are described in U.S. Pat. Nos. 3,036,036 and 4,210,741, both of which are incorporated by reference herein.

The polycarbonates of the invention may also be branched by condensing therein small quantities, e.g., 0.05 to 2.0 mol % (relative to the bisphenols) of polyhydroxyl compounds. Polycarbonates of this type have been described, for example, in German Offenlegungsschriften 1,570,533; 2,116,974 and 2,113,374; British Patents 885,442 and 1,079,821 and U.S. Pat. No. 3,544,514, which is incorporated herein by reference. The following are some examples of polyhydroxyl compounds which may be used for this purpose: phloroglucinol; 4,6-dimethyl-2,4,6-tri-(4-hydroxy-phenyl)-heptane; 1,3,5-tri-(4-hydroxyphenyl)-benzene; 1,1,1-tri-(4-hydroxyphenyl)-ethane; tri-(4-hydroxyphenyl)-phenyl-methane; 2,2-bis-[4,4-(4,4′-dihydroxydiphenyl)]-cyclohexyl-propane; 2,4-bis-(4-hydroxy-1-isopropylidine)-phenol; 2,6-bis-(2′-dihydroxy-5′-methylbenzyl)-4-methyl-phenol; 2,4-dihydroxybenzoic acid; 2-(4-hydroxy-phenyl)-2-(2,4-dihydroxy-phenyl)-propane and 1,4-bis-(4,4′-dihydroxytri-phenylmethyl)-benzene. Some of the other polyfunctional compounds are 2,4-dihydroxy-benzoic acid, trimesic acid, cyanuric chloride and 3,3-bis-(4-hydroxyphenyl)-2-oxo-2,3-dihydroindole.

In addition to the polycondensation process mentioned above, other processes for the preparation of the polycarbonates of the invention are polycondensation in a homogeneous phase and transesterification. The suitable processes are disclosed in U.S. Pat. Nos. 3,028,365; 2,999,846; 3,153,008; and 2,991,273 which are incorporated herein by reference.

The preferred process for the preparation of polycarbonates is the interfacial polycondensation process. Other methods of synthesis in forming the polycarbonates of the invention, such as disclosed in U.S. Pat. No. 3,912,688, incorporated herein by reference, may be used. Suitable polycarbonate resins are available in commerce, for instance, from Bayer MaterialScienice LLC under the MAKROLON trademark.

The compositions of the present invention contain a silver sodium zirconium phosphate of the following composition:

NaₓH_{y}Ag_{z}Zr₂(PO₄)₃ (I)

wherein x+y+z=1.
Such phosphates are marketed under the names of ALPHASAN RC 2000 (10% silver content) and ALPHASAN RC 5000 (3.8% silver content) by Milliken Chemical Co. Other known inorganic based silver-containing additives are based on soluble glass (Ishizuka Glass), zeolite (Agion Technologies), and ceramic (Sanitized AG) carriers.

The compositions preferably contain from 0.1 to 40 weight percent, more preferably from 0.5 to 30, most preferably from 1 to 15 weight percent, based on the weight of the composition of the antimicrobial compound. The antimicrobial compound may be present in the compositions of the present invention in an amount ranging between any combination of these values, inclusive of the recited values.

The inventive composition may contain additional functional components including mold release agents, colorants, hydrolytic stabilizers, radiation stabilizers, UV absorbers, antioxidants, surfactants, foaming agents, fillers, extenders, flame retardants and reinforcing agents.

The antimicrobial composition of the present invention is preferably prepared by mixing the antimicrobial agent with or without the additives noted above in the polycarbonate resin. The procedure and apparatus for making the composition are familiar to those skilled in the art.

The inventive stabilized compositions exhibit good rheology and color retention. The melt volume rate (“MVR”) of the compositions, according to ASTM 1238, varies by less than 40%, more preferaibly by less than 35%, and most preferably by less than 15%, from that of a comparable composition not containing the antimicrobial compound.

The present inventor speculates that the composition of the present invention may find use in preparing useful articles, such as medical articles, by any method of thermoplastic processing including injection molding and extrusion.

### EXAMPLES

The present invention is further illustrated, but is not to be limited, by the following examples. All quantities given in “parts” and “percents” are understood to be by weight, unless otherwise indicated. The following materials were used in the preparations of the examples:

| | |
|---|---|
| POLYCARBONATE A | a radiation stabilized polycarbonate available |
| | from Bayer MaterialScience as |
| | MAKROLON Rx1805; |
| POLYCARBONATE B | a linear polycarbonate available from Bayer |
| | MaterialScience as MAKROLON 3208; |
| POLYCARBONATE C | a linear polycarbonate available from Bayer |
| | MaterialScience as MAKROLON 3108; |
| POLYCARBONATE D | a linear polycarbonate available from Bayer |
| | MaterialScience as MAKROLON 2408; |
| ANTIMICROBIAL | available from Milliken under the |
| | ALPHASAN trade name; |
| CHAIN EXTENDER | available from BASF under the JONCRYL |
| | trade name; |
| SILICONE | a silicone oil available from Momentive |
| | Performance Materials as BAYSILONE OF |
| | EP 901; |
| RELEASE AGENT | available from Cognis under the LOXIOL |
| | trade name; |
| DYES | mixed blue and violet anthraquinone dyes; |
| POLYOL A | dihydropyran terminated polyether polyol; |
| | and |
| POLYOL B | a polypropylene glycol having a molecular |
| | weight of about 2,000 Da. |

Samples were prepared by twin screw extrusion and test specimens were injection molded using recommended polycarbonate molding conditions. Ash content was determined according to ASTM 5630. Melt volume rates were determined according to ASTM 1238 at 300° C., 1200 g load.

### Comparative Example 1 and Examples 2-7

Table I summarizes the amounts of components added in these examples and the resulting ash content and melt volume rates. As is apparent by reference to Table I, the melt volume rate increased in polycarbonate A with increasing amounts of silver antimicrobial additive.

TABLE I

| | Ex. C-1 | Ex. 2 | Ex. 3 | Ex. 4 | Ex. 5 | Ex. 6 | Ex. 7 |
|---|---|---|---|---|---|---|---|
| Component | | | | | | | |
| POLY- | 100 | 98.9 | 97.9 | 95.9 | 93.9 | 91.9 | 89.9 |
| CARBONATE | | | | | | | |
| A (%) | | | | | | | |
| ANTIMICROBIAL | 0 | 1 | 2 | 4 | 6 | 8 | 10 |
| (%) | | | | | | | |
| POLYOL A (%) | | 0.1 | 0.1 | 0.1 | 0.1 | 0.1 | 0.1 |
| Physical properties | | | | | | | |
| % Ash (pellets) | 0 | 0.98 | 1.86 | 3.72 | 5.68 | 7.88 | 9.81 |
| Melt volume rate | 6.7 | 7.7 | 9.0 | 11.9 | 12.7 | 12.5 | 14.1 |
| (cm³/10 min.) | | | | | | | |
| (pellets) | | | | | | | |
| Melt volume rate | 7.6 | 7.6 | 9.6 | 11.3 | 13.2 | 12.6 | 16.3 |
| (cm³/10 min.) (part) | | | | | | | |

### Comparative Example 8, and Examples 9-12

Table II summarizes the amounts of components added in these examples and the resulting ash content and melt volume rates, along with physical appearance observations. As can be appreciated by reference to Table II, the melt volume rate in polycarbonate B was stabilized even at high silver antimicrobial content with the elimination of material foaming and better color retention.

TABLE II

| | Ex. C-8 | Ex. 9 | Ex. 10 | Ex. 11 | Ex. 12 |
|---|---|---|---|---|---|
| Component | | | | | |
| POLYCARBONATE B | 99.3988 | 90 | 89.3988 | 89.2988 | 89.2988 |
| POLYOL A | 0.6 | — | 0.6 | 0.6 | 0.6 |
| ANTIMICROBIAL | 0 | 10 | 10 | 10 | 10 |
| DYES | 0.0012 | — | 0.0012 | 0.0012 | 0.0012 |
| SILICONE | — | — | — | 0.1 | — |
| CHAIN EXTENDER | — | — | — | — | 0.1 |
| Physical properties | | | | | |
| Ash content (%) | 0.04 | 9.66 | 9.75 | 9.57 | 9.43 |
| Melt volume rate | 6.6 | 6.4 | 12.1 | 4.6 | 9.1 |
| (cm³/10 min.) (pellets) | | | | | |
| Appearance of pellet | purple, | grayish, | brown, | purplish, | brown, |
| melt extrudate | clear strand | translucent | translucent | translucent | translucent |
| | | strand | foamy strand | strand | foamy strand |
| Melt volume rate | 7.8 | 7.0 | 12.8 | 5.1 | 5.1 |
| (cm³/10 min.) (part) | | | | | |
| Appearance of part melt | purple, | tan, | brown, | purplish, | brown, |
| extrudate | clear strand | translucent | translucent | translucent | translucent |
| | | strand | foamy strand | strand | strand |

### Comparative Example 13 and Examples 13-18

Table III summarizes the amounts of components added in these examples and the resulting ash content and melt volume rates. As is apparent by reference to Table III, the melt volume rate in all three of the polycarbonates examined was stabilized with the silver antimicrobial content.

TABLE III

| | Ex. C-13 | Ex. 14 | Ex. 15 | Ex. 16 | Ex. 17 | Ex. 18 |
|---|---|---|---|---|---|---|
| Component | | | | | | |
| POLYCARBONATE B | 99.30 | 98.30 | 97.30 | — | — | — |
| POLYCARBONATE C | — | — | — | 97.30 | — | — |
| POLYCARBONATE D | — | — | — | | 97.30 | 97.05 |
| ANTIMICROBIAL | — | 1.00 | 2.00 | 2.00 | 2.00 | 2.00 |
| RELEASE AGENT | — | — | — | — | — | 0.25 |
| SILICONE | 0.10 | 0.10 | 0.10 | 0.10 | 0.10 | 0.10 |
| POLYOL B | 0.60 | 0.60 | 0.60 | 0.60 | 0.60 | 0.60 |
| Physical properties | | | | | | |
| Melt volume rate | 5.4 | 5.3 | 5.2 | 7.0 | 26.0 | 26.6 |
| (cm³/10 min.) | | | | | | |
| Ash content (%) | 0.0 | 1.0 | 2.0 | 2.1 | 2.0 | 2.0 |
| Appearance of pellet | clear | colorless, | colorless, | colorless, | colorless, | colorless, |
| melt extrudate | strand | translucent | translucent | translucent | translucent | translucent |
| | | strand | strand | strand | strand | strand |

The foregoing examples of the present invention are offered for the purpose of illustration and not limitation. It will be apparent to those skilled in the art that the embodiments described herein may be modified or revised in various ways without departing from the spirit and scope of the invention. The scope of the invention is to be measured by the appended claims.

1. A stabilized thermoplastic molding composition comprising an aromatic polycarbonate resin, 0.1 to 2.0 weight percent, based on the weight of the composition, of a polyol, and from about 0.1 to about 40 weight percent, based on the weight of the composition, of a silver sodium zirconium phosphate antimicrobial compound of the formula (I)
NaₓH_{y}Ag_{z}Zr₂(PO₄)₃ (I)
wherein x+y+z=1, and
wherein the composition has a melt volume rate according to ASTM 1238 within about 40% of the melt volume rate of a comparable composition not containing the antimicrobial compound.

2. The composition according to claim 1, wherein the antimicrobial compound is present in an amount of from about 0.5 to about 30 weight percent.

3. The composition according to claim 1, wherein the antimicrobial compound is present in an amount of from about 1 to about 15 weight percent.

4. The composition according to claim 1, wherein the melt volume rate of the composition is within about 35% of a comparable composition not containing the antimicrobial compound.

5. The composition according to claim 1, wherein the melt volume rate of the composition is within about 15% of a comparable composition not containing the antimicrobial compound.

6. An article of manufacture comprising the composition according to claim 1.

7. A process for the production of a stabilized molding composition comprising combining an aromatic polycarbonate resin, 0.1 to 2.0 weight percent, based on the weight of the composition, of a polyol, and from about 0.1 to about 40 weight percent, based on the weight of the composition, of a silver sodium zirconium phosphate antimicrobial compound of the formula (I)
NaₓH_{y}Ag_{z}Zr₂(PO₄)₃ (I)
wherein x+y+z=1, and
wherein the composition has a melt volume rate according to ASTM 1238 within about 40% of the melt volume rate of a comparable composition not containing the antimicrobial compound.

8. The process according to claim 7, wherein the antimicrobial compound is present in an amount of from about 0.5 to about 30 weight percent.

9. The process according to claim 7, wherein the antimicrobial compound is present in an amount of from about 1 to about 15 weight percent.

10. The process according to claim 7, wherein the melt volume rate of the composition is within about 35% of a comparable composition not containing the antimicrobial compound.

11. The process according to claim 7, wherein the melt volume rate of the composition is within about 15% of a comparable composition not containing the antimicrobial compound.

12. An article of manufacture comprising the composition made according to the process of claim 7.

13. The composition according to claim 1, wherein the polyol is selected from the group consisting of a dihydropyran terminated polyether polyol and a polypropylene glycol having a molecular weight of about 2,000 Da.

14. The process according to claim 7, wherein the polyol is selected from the group consisting of a dihydropyran terminated polyether polyol and a polypropylene glycol having a molecular weight of about 2,000 Da.
